(19) United States
(12) Patent Application Publication
(10) Pub. No.: US 2006/0216273 A1
(43) Pub. Date: Sep. 28, 2006

(54) Gene therapy for colorectal cancer

(76) Inventors: Min-Chuan Huang, Jhubei City (TW); Hsiao-Yu Chen, Danshui Town (TW)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(21) Appl. No.: 11/134,938

(22) Filed: May 23, 2005

Related U.S. Application Data
(60) Provisional application No. 60/665,437, filed on Mar. 25, 2005.

(51) Int. Cl. A61K 48/00

(52) U.S. Cl. 424/93.2; 514/44

(57) ABSTRACT

The present invention relates to the novel use of a core 2 β-1,6-N-acetylglucosaminyltransferase (C2GnT) in the treatment of colorectal cancer. Accordingly, the present invention provides a method for treating colorectal cancer in a subject including administering to the subject a nucleic acid molecule including a sequence encoding C2GnT, wherein the sequence is expressed in cancer cells. The present invention also provides a pharmaceutical composition for treating colorectal cancer including a nucleic acid molecule including a sequence encoding C2GnT, and a pharmaceutically acceptable carrier.

### CROSS-REFERENCE TO RELATED APPLICATION

This application is entitled to priority pursuant to 35 U.S.C. § 119(e) to U.S. Provisional Patent Application No. 60/665,437, filed Mar. 25, 2005, the disclosure of which is hereby incorporated herein by reference.

### BACKGROUND OF THE INVENTION

The present invention relates generally to a method for treating colorectal cancer.

Colorectal cancer is one of the most common cancers in industrialized countries. Nearly half of the patients with colorectal cancer still die of metastatic diseases after curative surgery. Until now, the thymidylate-synthase inhibitor 5-fluorouracil (5-FU), which was developed over forty years ago, is still the most commonly used drug in the chemotherapy for colorectal cancer.

Numerous schedules of 5-FU administration have been investigated over the last years, comparing low doses versus high doses, and bolus versus continuous infusions. However, even after all this time, there is still no standard schedule for 5-FU administration recognized by the oncology profession. In fact, all the regimens of 5-FU have the disadvantages of the requirement of intravenous administration and the possible negative impact of prolonged infusions on life quality.

Currently some oral compounds are under development for the treatment of colorectal cancer, such as the pyrimidine analogues doxofluridine, capecitabine and carmofur, and the camptothecin derivatives irinotecan, 9-amino camptothecin and rubitecan. However, the dosage of these oral compounds is limited due to their toxicities.

Many physiological processes require cell-cell and/or cell-extracellular matrix interactions. Such adhesion events may be required for cell activation, migration, proliferation and differentiation. Cell-cell and cell-matrix interactions are mediated through several families of cell adhesion molecules (CAMs) including the selecting, integrins, cadherins and immunoglobulin superfamily proteins. It is known that CAMs play an essential role in both normal and pathophysiological processes, including cancer metastasis.

Tumor progression is involved in altered (1) cell-cell and (2) cell-substratum attachment, and (3) cell migration and invasion through basement membranes, thereby releasing tumor cells into the circulation or lymphatic system. These three processes are probably mediated by different receptors (Zhang, Z. et al., 1993, J. Cell Biol. 122: 235-242). Each process thus forms a different step of the metastatic cascade and a combination of all three is likely to be required for metastasis to occur. The various cell adhesion molecule families probably act in conjunction during the metastatic processes.

Sialylated fucosylated lactosamines, a specific class of carbohydrates expressed on the cell surface, are critical components of ligands for the aforementioned CAM selectin. They are present in a variety of mucin-type glycoproteins that contain (O)-linked oligosaccharides, or O-glycans. Sialylated fucosylated lactosamines are formed by modifications to oligosaccharides having the branched core 2 structure Gal[β] 1,3[GlcNAc[β] 1,6]GalNAc[α]-O, which is derived from the core 1 structure Gal[β] 1,3GalNAc[α]-O by the action of a core 2 β-1,6-N-acetylglucosaminyltransferase (C2GnT). Previous reports have established C2GnT as a critical enzyme in O-glycan biosynthesis.

### BRIEF SUMMARY OF THE INVENTION

The present invention is directed to a gene therapy for colorectal cancer that obviates one or more problems resulting from the limitations and disadvantages of the prior art.

In accordance with an embodiment of the present invention, there is provided a method for treating colorectal cancer in a subject, including humans and other mammals, comprising administering to the subject a nucleic acid molecule comprising a sequence encoding C2GnT, wherein C2GnT exhibits core 2 branching activity on O-glycans. The sequence is expressed in cancer cells.

Also in accordance with the present invention, there is provided a pharmaceutical composition for treating colorectal cancer comprising a nucleic acid molecule comprising a sequence encoding C2GnT, and a pharmaceutically acceptable carrier.

Further in accordance with the present invention, there is provided a nucleic acid molecule comprising a sequence encoding C2GnT for use in the treatment of colorectal cancer.

Still in accordance with the present invention, there is provided the use of a nucleic acid molecule comprising a sequence encoding C2GnT in the manufacture of a medicament for the treatment of colorectal cancer.

Additional features and advantages of the present invention will be set forth in part in the description which follows, and in part will be apparent from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one embodiment of the present invention and together with the description, serve to explain the principles of the invention.

### BRIEF DESCRIPTION OF THE SEVERAL VIEWS OF THE DRAWINGS

Reference will now be made in detail to the present embodiment of the invention, an example of which is illustrated in the accompanying drawings.

In the drawings:

FIG. 1 comprises FIGS. 1A and 1B. FIG. 1A is an image that shows the effect of hC2GnT-M (identified below) expression on the morphology of HCT116 human colon carcinoma cells. Cells were grown onto 10-cm culture plates (Falcon). After seeding for 24 h, cells were photographed under a phase-contrast microscope. FIG. 1B is a graph that shows the percentage of round cells that were calculated at five different fields and presented as mean values±S.D.

FIG. 2 is a graph that shows the effect of hC2GnT-M expression on the colony-forming ability of HCT116 cells. Colony formation assay in soft agar was performed with 1×10⁴ transfectants in 10 cm dishes. The number of colonies was counted and shown as indicated.

FIG. 3 is a graph that shows the effect of hC2GnT-M expression on the growth rate of HCT116 cells. 4×10⁴ stable hC2GnT-M and mock transfectants were seeded into each well of 24-well plates for 3 days and then 6-well plates for another 3 days. Cell number was counted every 24 h. The number of cells shown in each time point represents the mean of three independent experiments, ±S.D.

FIG. 4 is a graph that shows the effect of hC2GnT-M expression on the apoptosis of HCT116 cells. Flow cytometry with propidium iodide staining was performed using stable transfectants. Cells were seeded in 6-well plates and cultured for 24 h. Cells in suspension and adherent to substratum were collected and then fixed with 70% ice-cold ethanol. After staining with propidium iodide, DNA content was analyzed using a flow cytometer. The sub-G1 population was considered as apoptotic cells and shown as indicated.

FIG. 5 are graphs comprising FIGS. 5A and 5B. FIG. 5A shows the effect of hC2GnT-M expression on the adhesion of HCT116 cells to substratum. 4×10⁴ of transfectants were resuspended in 100-μl DMEM with or without 10% FBS and then seeded into each well of 96-well plates, and incubated at 37° C. for 1 h. Non-specific binding cells were carefully removed and adherent cells were counted under microscope. FIG. 5A shows the number of cells counted with serum, while FIG. 5B shows the number of cells counted without serum.

FIG. 6 shows the effect of hC2GnT-M expression on the migration of HCT116 cells. Wound healing assays were performed using stable hC2GnT-M and mock HCT116 transfectants. Confluent cells were scraped with a 250 μl tip and observed every 6 hours. Representative images were shown at the indicated time. The velocity of cell migration was presented as migration distance (μm)/time (h).

FIG. 7 comprises FIGS. 7A and 7B. FIG. 7A is an image that shows the effect of hC2GnT-M expression on the metastasis of HCT116 cells. Matrigel invasion assays were performed using stable hC2GnT-M and mock HCT116 transfectants. 2×10⁵ cells were seeded in each chamber and cultured for 48 h. Invaded cells were stained with 0.5% crystal violet and counted under a microscope. FIG. 7B is a graph that shows the number of cells counted.

FIG. 8 comprises FIGS. 8A through 8D and relates to the effect of hC2GnT-M on the suppression of tumor growth in vivo. 8×10⁶ stable mock or hC2GnT-M HCT116 transfectants were injected subcutaneously into six 6-week-old female BALB/c nude mice for each group. FIG. 8A is a graph that shows average tumor volumes of each group at different time points after injection are shown. FIG. 8B comprises images of tumors grown on the back of nude mice. FIG. 8C comprises images of tumors excised from the mice on day 20 post injection (two mice of the hC2GnT-M group had no tumor). FIG. 8D is a graph showing average tumor weight of each group on day 20 post injection.

### DETAILED DESCRIPTION OF THE INVENTION

It has now been found that introducing a nucleic acid molecule comprising a sequence encoding C2GnT into colorectal cancer cells and subsequently expressing the encoded C2GnT in the cells, can reverse the cancerous phenotype of the cells. Therefore, the present invention is directed to the use of a nucleic acid molecule comprising a sequence encoding C2GnT in the manufacture of a medicament for the treatment of colorectal cancer.

As used herein, the term “C2GnT” not only refers to the C2GnT protein per se but also preferably includes fragments, variants, derivatives and analogues thereof. A fragment, variant, derivative or analogue may differ from the C2GnT protein in amino acid sequence by one or more substitutions, additions, deletions, fusions and truncations, which may be present in any combination. Such substitutions, additions, deletions, fusions and truncations are deemed to be within the scope of those skilled in the art from the teachings herein. In any event, the fragment, variant, derivative or analogue should retain substantially the same biological function or activity as the C2GnT protein.

In one aspect, the present invention provides a method for treating colorectal cancer in a subject comprising administering to the subject a nucleic acid molecule comprising a sequence encoding C2GnT, wherein the sequence is expressed in cancer cells.

The nucleotide sequence encoding C2GnT utilized in the present invention may be derived from any origin or species. Preferably, the nucleotide sequence is derived from human species. Three C2GnT proteins have been identified in human species, namely C2GnT-L (Amino Acid SEQ ID NO:2) (also known as C2GnT1), C2GnT-M (Amino Acid SEQ ID NO:4 or 6) (also known as C2GnT2 (Amino Acid SEQ ID NO:4) or C2/4GnT (Amino Acid SEQ ID NO:6)) and C2GnT-T (Amino Acid SEQ ID NO:8) (also known as C2GnT3), and their sequences can be found in the GenBank with the accession number M97347 (Nucleotide—SEQ ID NO:1; Amino Acid—SEQ ID NO:2), AF038650 (Nucleotide—SEQ ID NO:3; Amino Acid—SEQ ID NO:4)/AF102542 (Nucleotide—SEQ ID NO:5; Amino Acid—SEQ ID NO:6), and AF132035 (Nucleotide—SEQ ID NO:7; Amino Acid—SEQ ID NO:8), respectively (Schwientek, T. et al., 2000, J. Biol. Chem. 275: 11106-11113). Most preferably, the nucleotide sequence of the present invention encodes human C2GnT-M (Amino Acid SEQ ID NO: 4 or 6).

As described by Schwientek, T., et al., (2000), supra, the three known C2GnT members, C2GnT-L, C2GnT-M and C2GnT-T, all exhibit core 2 branching enzyme activity on O-glycans. From the sequence similarity analysis, high amino acid sequence similarity with several conserved motifs was found in the putative catalytic domains of the three proteins. In addition, these three proteins contain nine conserved cysteines, and there is one conserved potential N-linked glycosylation site located in their stem region.

The nucleic acid molecule of the present invention may be in the form of DNA, cDNA or RNA, such as mRNA obtained by cloning or produced by chemical synthetic techniques. The DNA may be single or double stranded. Single stranded DNA may be the coding or sense strand, or it may be the non-coding or anti-sense strand. For therapeutic use, the nucleic acid molecule should be in a form capable of being expressed to produce a functional C2GnT protein in the cancer cells in the subject to be treated.

Preferably in gene therapy, the nucleic acid molecule is administered such that it is expressed in the subject to be treated, for example in the form of a recombinant DNA molecule comprising a sequence encoding C2GnT operatively linked to a sequence which controls expression, such as in an expression vector. Such a vector will thus include appropriate transcriptional control signals including a promoter region capable of expressing the coding sequence, when the promoter is operable in the subject to be treated. Thus for human gene therapy, the promoter, which term includes not only the sequence necessary to direct RNA polymerase to the transcriptional start site, but also, if appropriate, other operating or controlling sequences including enhancers, is preferably a human promoter sequence from a human gene, or from a gene which is typically expressed in humans, such as the promoter from human cytomegalovirus (CMV). Among known eukaryotic promoters suitable in this regard is the CMV immediate early promoter, the HSV thymidine kinase promoter, the early and late SV40 promoters, the promoters of retroviral long terminal repeats (LTRs), such as those of the Rous sarcoma virus (“RSV”), and metallothionein promoters, such as the mouse metallothionein-1 promoter. The expression vectors may also include selectable markers, such as for antibiotic resistance, which enable the vectors to be propagated.

The appropriate nucleotide sequence may be inserted into the vector by any of a variety of well-known and routine techniques, such as, for example, those set forth in Sambrook et al., MOLECULAR CLONING, A LABORATORY MANUAL, 2nd Ed.; Cold Spring Harbor Laboratory Press, Cold Spring Harbor, N.Y. (1989).

Vectors that can be used in the present invention include, but are not limited to: adenovirus vectors, adeno-associated virus vectors, Epstein-Barr virus vectors, Herpes virus vectors, attenuated HIV vectors, retroviral vectors, and vaccinia virus vectors.

Methods to introduce a nucleic acid molecule into cells have been well known in the art. A naked nucleic acid molecule may be introduced into the cell by direct transformation. Alternatively, the nucleic acid molecule may be embedded in liposomes. Other physical, mechanical or electrical methods may also be utilized, such as particle bombardment (also known as the “gene gun” technology), ultrasound, electrical stimulation, electroporation and microseeding.

In a further aspect, the present invention provides a pharmaceutical composition for treating colorectal cancer comprising a nucleic acid molecule comprising a sequence encoding C2GnT, and a pharmaceutically acceptable carrier.

The term “pharmaceutically acceptable carrier” as used herein encompasses any of the standard pharmaceutical carriers. Such carriers may include, but are not limited to: saline, buffered saline, dextrose, water, glycerol, ethanol and combinations thereof.

The pharmaceutical composition of the present invention may be constituted into any form suitable for the mode of administration selected. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The effect of the present invention can be well understood from the following experimental examples, which are intended to be a way of illustrating but not limiting the present invention.

### EXAMPLES

### Example 1

### Construction of Plasmid

RT-PCR was performed to clone full length human mucin-type core 2 β-1,6-N-acetylgucosaminyltransferase (hC2GnT-M) (GenBank accession number AF-102542) from normal colon total RNA (Clontech™, Becton, Dickinson and Company). The sense primer was 5′-cggaattcGACGATGGTTCAATGGAA-3′ (SEQ ID NO:9), and the antisense primer was 5′-gctctagagccAAGTTCAGTCCCAT-3′ (SEQ ID NO:10). The RT-PCR products were cloned into pcDNA™3.1/Myc-His A (InVitrogen Corp.) to generate a hC2GnT-M/Myc-His fusion gene. The insert was confirmed by DNA sequencing.

### Example 2

### Cell Culture and Transfection

HCT116 (BCRC60349) was purchased from Bioresources Collection and Research Center (BCRC), Taiwan, and maintained with DMEM containing 10% FBS in humidified tissue culture incubator, 37° C., 5% CO₂ atmosphere. Four μg of hC2GnT-M/pcDNA™3.1/Myc-His A and pcDNA™3.1/Myc-His A (mock) were transfected into 5×10⁵ cells of HCT116 using Lipofectamine™ 2000 (InVitrogen Corp.). After 24 hours of transfection, the cells were trypsinized and plated on three 100-mm dishes to form colonies and cultured with 10% fetal bovine serum-DMEM containing 600 μg/ml of G418. After 2 weeks selection, G418-resistant clones were isolated and transferred to 24-well plates. The positive clones and expression levels of hC2GnT-M were confirmed by using RT-PCR and immunocytochemistry of anti-myc antibodies.

### Example 3

### Expression of hC2GnT-M Changes Cell Morphology

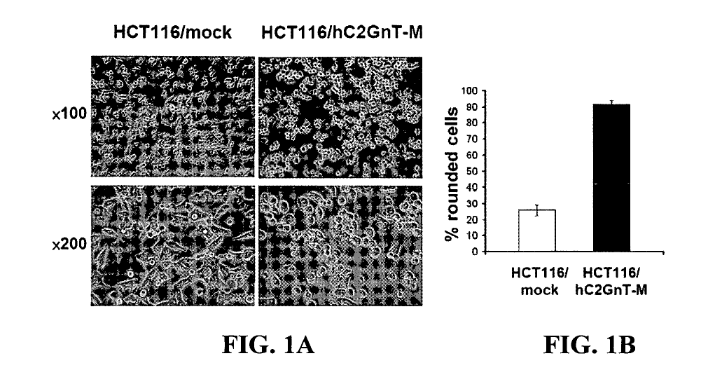

The transfected HCT116 Cells were grown onto 10-cm culture plates (Falcon™, Becton, Dickinson and Company). After seeding for 24 h, cells were photographed under a phase-contrast microscope. Round cells were calculated at five different fields and presented as mean values±SD (FIG. 1B).

The number of round cells was 26±3 per field for the mock-transfected cells. However, the number of round cells for hC2GnT-M transfected cells increased to 92±3 per field, which is 3.5 fold compared to the mock-transfected cells. This demonstrated that hC2GnT-M inhibited cancer cell spreading and caused cancer cells round up.

### Example 4

### Expression of hC2GnT-M Inhibits Colony-forming Ability

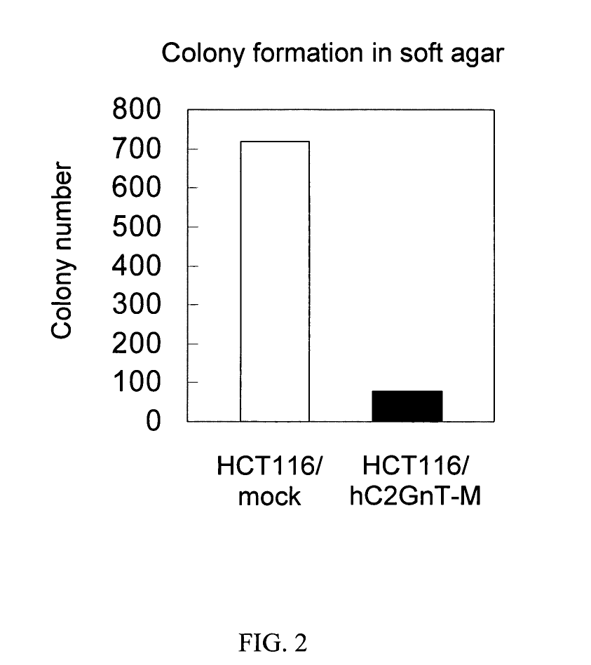

Colony formation assay in soft agar was performed to investigate the effect of hC2GnT-M expression on the colony-forming ability of colon cancer cells. 1×10⁴ transfected HCT116 cells in 0.3% Bactoagar in DMEM supplemented with 10% FBS were overlaid on a base of 0.6% Bactoagar in DMEM supplemented with 10% FBS in 10-cm dishes. Cells were incubated at 37° C., 5% CO₂ atmosphere. The number of colonies was counted at the end of 3 weeks (FIG. 2).

It is well established that colony formation assay in soft agar is predictive of tumorigenicity in vivo. hC2GnT-M stable transfectants inhibited colony-formation ability of HCT116 colon cancer cells by 89% as compared with mock stable transfectants. In addition, the average colony size of hC2GnT-M stable transfectants is smaller than the mock stable transfectants.

### Example 5

### Expression of hC2GnT-M Inhibits Cell Growth

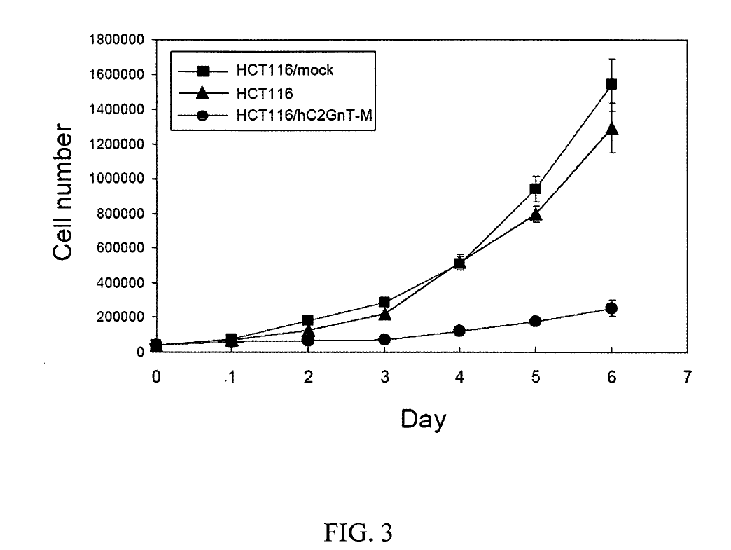

To analyze the effect of hC2GnT-M expression on cell growth, 4×10⁴ of stable hC2GnT-M and mock HCT116 transfectants were seeded to 24-well plates for 3 days and then 6-well plates for another 3 days. Triplicate wells were plated for each time point, which was taken at 24 h intervals for 6 days. Cell number in a monolayer was counted every 24 hours to determine the cell growth rate, and the results of counts were used to plot the growth curve with standard error (FIG. 3).

The cell number of stable hC2GnT-M transfectants was only 16.5% of mock transfectants on day 6. This data showed that hC2GnT-M inhibited the cell growth of colon cancer cells.

### Example 6

### Expression of hC2GnT-M Causes Cell Death

To analyze whether the reduced cell growth was due to an inhibition of cell cycle progression or an increase in cell death, we performed flow cytometry on stable hC2GnT-M and mock HCT116 transfectants with propidium iodide, a fluorescent dye that binds to DNA as described (Wang, N. S., et al., 2001, J. Biol. Chem. 276(47): 44117-28). The hC2GnT-M and mock transfectants were trypsinized and washed once with PBS. Cells were fixed with ice-cold 70% ethanol for 30 minutes. The cells were then treated with 200 μg/ml RNase A and 50 μg/ml propidium iodide for 30 min. at 37° C. The stained cells were analyzed using Flow Cytometer (BD Biosciences). Cells with DNA content less than the G₁ amount of untreated cells were considered apoptotic.

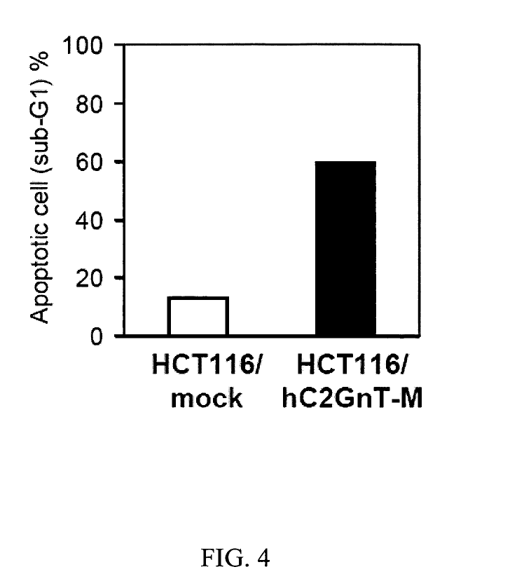

A hallmark of apoptotic cell death is the enzymatic cleavage of chromosomal DNA by endonucleases activated by the caspase cascade. The degradation of chromosomal DNA of apoptotic cells results in a decrease in total DNA content to a level that is lower than the DNA content of cells in G1 phase. Consequently, the DNA content of apoptotic cells appears in a DNA content profile as a peak below the DNA content of cells in G1 phase. We determined that the population of sub-G1 cells of hC2GnT-M-transfectants and mock-transfectants was 13.03% and 59.61%, respectively (FIG. 4). A significant increase in the sub-G1 DNA fraction was observed for stable hC2GnT-M transfectants, suggesting that expression of hC2GnT-M leads to an increase in DNA fragmentation and apoptotic cell death in colon cancer cells.

### Example 7

### Expression of hC2GnT-M Blocks Cell Adhesion

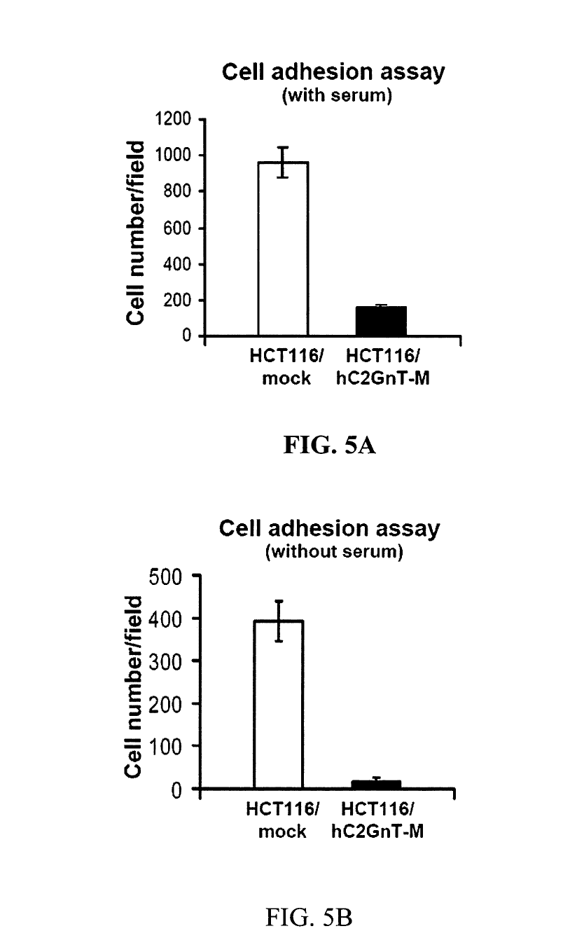

For the cell adhesion assay, transfected HCT116 cells were trypsinized and resuspended in DMEM with or without 10% FBS. 4×10⁴ cells were replated into each well of 96-well culture dishes and allowed to adhere for 1 h at 37° C. Non-adherent cells were removed by washing twice with PBS, after which the adherent cells from five wells were photographed at 100× magnification under a phase contrast microscope and counted (FIG. 5A). The mean values±SD were presented (FIG. 5B).

Over-expression of the C2GnT-M diminished cell adhesion of HCT116 by 84% as stable transfectants were resuspended in DMEM containing 10% FBS. Furthermore, cell adhesion was decreased by up to 96% as the cells were resuspended in serum-free DMEM. Apparently, the hC2GnT-M was able to significantly block cell adhesions under conditions with or without serum.

### Example 8

### Expression of hC2GnT-M Reduces Cell Migration

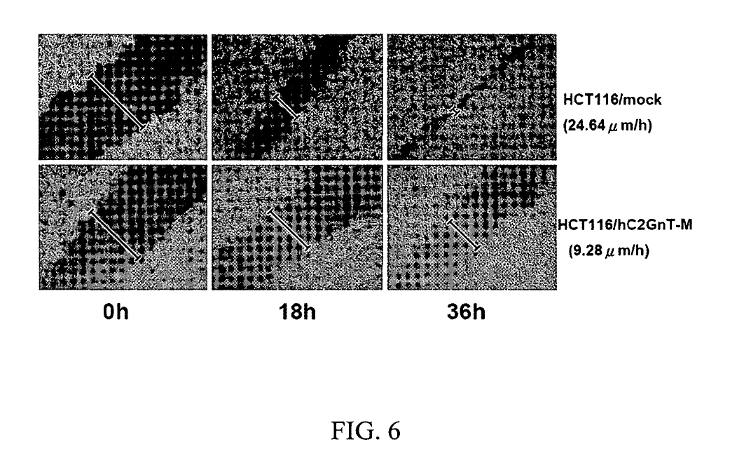

The effect of hC2GnT-M expression on cell migration was assessed by the monolayer wound healing assay. Transfected HCT116 cells were seeded on 12-well culture dishes and grown until confluence in DMEM supplemented with 10% FBS. The monolayer was scratched with a sterile 250-μl pipette tip to create a denuded area, and then the cells at the wound edges were allowed to migrate into the denuded area over a 6 h period. The migration of cells was inspected under a microscope. Pictures were taken directly at the time of scratching and after scratching at 6 h intervals (FIG. 6). Migration velocity was presented as migration distance (μm)/time (h).

The migration velocity of mock transfectants and hC2GnT-M transfectants was 24.64 μm/h and 9.28 μm/h, respectively. It is evident that the cells expressing hC2GnT-M migrated more slowly into the wound area than the cells expressing the vector alone. This data showed that hC2GnT-M decreased the cell motility of colon cancer cells.

### Example 9

### Expression of hC2GnT-M Inhibits Cell Invasion

Tumor cell invasion is the critical process of cancer metastasis. Carcinoma invasion involves cell attachment to the extracellular matrix (ECM), localized degradation of the ECM, and cell migration through the tissue barrier (Liotta, L. A. and Stetler-Stevenson, W. G., 1991, Cancer Res. 51(18 Suppl): 5054s-5059s). To investigate the role of hC2GnT-M on invasion of colorectal cancer, stable hC2GnT-M and mock HCT116 transfectants were analyzed by Matrigel invasion assay, which mimics active transmigration of tumor cells across a reconstituted basement membrane.

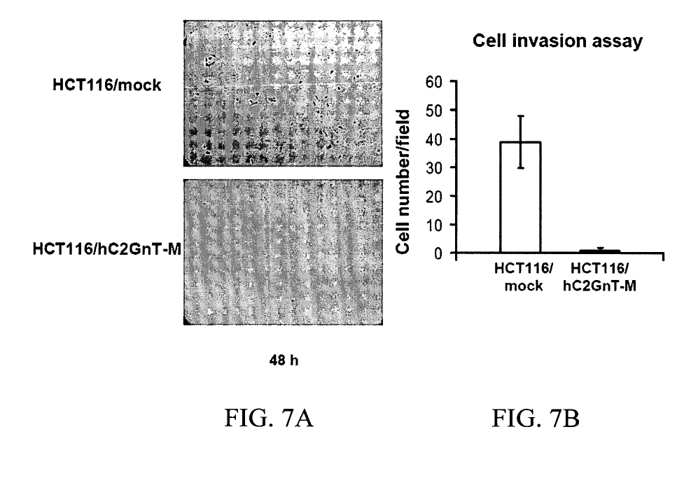

Matrigel invasion assay was preformed using a BioCoat™ Matrigel™ Invasion Chamber (Becton, Dickinson and Company) according to the protocol provided by the manufacturer. Briefly, 2×10⁵ cells in 500 μl DMEM were added to each chamber and allowed to invade Matrigel for 42 hours in humidified tissue culture incubator, 37° C., 5% CO₂ atmosphere. The non-invading cells on the upper surface of the membrane were removed from the chamber, and the invading cells on the lower surface of the membrane were stained with 0.5% crystal violet. After two washes with distilled water, the chambers were allowed to air dry. The number of invading cells per field was counted under a phase contrast microscope (FIG. 7A). The mean values±SEM were calculated from the numbers of five different fields of the microscope and are graphed in FIG. 7B.

The invaded cell numbers of HCT116 cells stably expressing C2GnT-M were 39±9 per field. In sharp contrast, only 1±1 cells/field was observed in mock-transfected control cells. These data showed that hC2GnT-M expression resulted in almost complete inhibition of invasion as determined by the Matrigel invasion assay.

### Example 10

### hC2GnT-M Suppresses Tumor Growth In Vivo

The effect of hC2GnT-M gene on tumor growth in vivo was evaluated by subcutaneous injection of stable hC2GnT-M transfectants into nude mice. For tumorigenicity assay in vivo, 8×10⁶ of stable mock or hC2GnT-M HCT116 transfectants were injected subcutaneously into 6-week-old female BALB/c nude mice. Cells were suspended in PBS at a concentration of 8×10⁷ cells/ml, and injected into six mice for each group. Tumor volumes were measured at different time points during 20 days with calipers, and calculated by the following formula: Volume=0.4×A×B² where A is the larger and B is the smaller axis (Ovadia et al., 1975, Eur. J. Cancer 11: 413-417). On day 20 post injection, the mice were sacrificed and the tumors were excised from the mice and photographed.

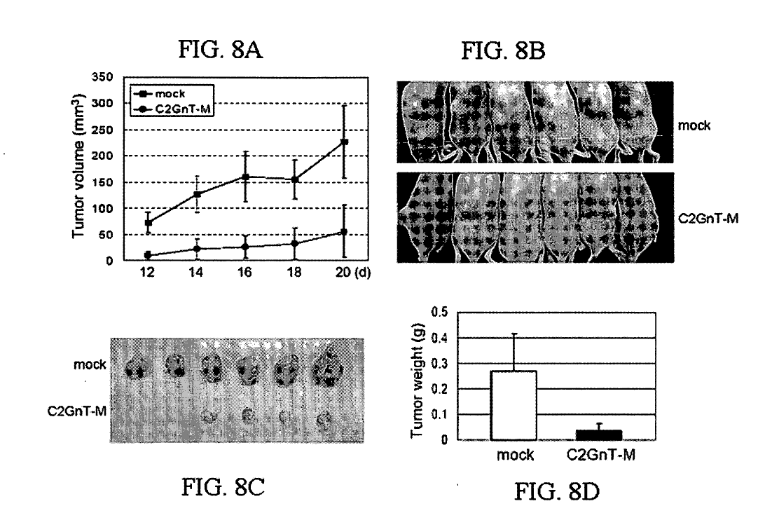

The results were shown in FIG. 8. The mock transfectants formed large tumors within a short period of time. In contrast, the hC2GnT-M transfectants exhibited significant suppression of tumor weight, reaching only 13% of the control xenografts by day 20. Furthermore, two mice with hC2GnT-M xenograft did not show tumor growth when they were sacrificed. These data indicated that the expression of hC2GnT-M cDNA in HCT116 cells resulted in significant suppression of tumor growth.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

We claim:

1. A method for treating colorectal cancer in a subject comprising administering to the subject a nucleic acid molecule comprising a sequence encoding C2GnT, wherein C2GnT exhibits core 2 branching enzyme activity on O-glycans.

2. The method according to claim 1, wherein C2GnT is selected from the group consisting of C2GnT-L, C2GnT-M and C2GnT-T.

3. The method according to claim 1, wherein the nucleic acid molecule is a vector.

4. The method according to claim 3, wherein the vector is selected from the group consisting of an adenovirus vector, an adeno-associated virus vector, an Epstein-Barr virus vector, a Herpes virus vector, an attenuated HIV vector, a retroviral vector, and a vaccinia virus vector.

5. The method according to claim 1, wherein the sequence encodes human C2GnT.

6. The method according to claim 3, wherein the sequence encodes human C2GnT.

7. The method according to claim 4, wherein the sequence encodes human C2GnT.

8. The method according to claim 5, wherein the human C2GnT is C2GnT-M (SEQ ID NO:4 or 6).

9. The method according to claim 5, wherein the human C2GnT is C2GnT-M (SEQ ID NO:4 or 6).

10. The method according to claim 6, wherein the human C2GnT is C2GnT-M (SEQ ID NO:4 or 6).

11. A pharmaceutical composition for treating colorectal cancer comprising a nucleic acid molecule comprising a sequence encoding C2GnT, and a pharmaceutically acceptable carrier.

12. The pharmaceutical composition according to claim 11, wherein the nucleic acid molecule is a vector.

13. The pharmaceutical composition according to claim 12, wherein the vector is selected from the group consisting of an adenovirus vector, an adeno-associated virus vector, an Epstein-Barr virus vector, a Herpes virus vector, an attenuated HIV vector, a retroviral vector, and a vaccinia virus vector.

14. The pharmaceutical composition according to claim 11, wherein the sequence encodes human C2GnT.

15. The pharmaceutical composition according to claim 12, wherein the sequence encodes human C2GnT.

16. The pharmaceutical composition according to claim 13, wherein the sequence encodes human C2GnT.

17. The pharmaceutical composition according to claim 14, wherein the human C2GnT is C2GnT-M (SEQ ID NO:4 or 6).

18. The pharmaceutical composition according to claim 15, wherein the human C2GnT is C2GnT-M (SEQ ID NO:4 or 6).

19. The pharmaceutical composition according to claim 16, wherein the human C2GnT is C2GnT-M (SEQ ID NO:4 or 6).


SEQUENCE LISTING (10 sequences)

SEQ ID NO: 1 - DNA, 2110 bp, Homo sapiens
  CDS (220)..(1506)
  ref <308>: DATABASE ACCESSION NUMBER: GenBank / M97347
  ref <309>: DATABASE ENTRY DATE: 1993-04-27
  ref <313>: RELEVANT RESIDUES: (1)..(2110)
gtgaagtgctcagaatggggcaggatgtcacctggaatcagcactaagtgattcagactttccttacttttaaatgtgctgctcttcatttcaagatgccgttgcagctctgataaatgcaaactgacaaccttcaaggccacgacggagggaaaatcattggtgcttggagcatagaagactgcccttcacaaaggaaatccctgattattgtttgaaatgctgaggacgttgctgcgaaggagacttttttcttatcccaccaaatactactttatggttcttgttttatccctaatcaccttctccgttttaaggattcatcaaaagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcgttcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttgtggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctatctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgagacacaaagctttggagacattaaaacactgaccattacgggcaattttatgaacaagaagaaggatacacaaaacgtaccttatctgtttccccttccttgtcagcgtcgggaagatggtatgaagtcctctttggggcagggactctagtagatcttcttgtcagagaagctgcatggtttctgcagagcacagttagctagaaaggtgatagcattaaatgttcatctagagttaatagtgggaggagtaaaggtagccttgaggccagagcaggtagcaaggcattgtggaaagaggggaccagggtggctggggaagaggccgatgcataaagtcagcctgttccaagtgctcagggacttagcaaaatgagaagatgtgacctgtgccaaaactattttgagaattttaaatgtgaccatttttctggtatgccaataaaddcttacagcaacaaataatcaaagatacaattaatctgatattatatttgttgaaatagaaatttgattgtactataaatgatttttgtaaataatttatattctgctctaatactgtactgtgtagtgtgtctccgtatgtcatctcagggagcttaaaatgggcttgatttaacattgaaaaaaaat

SEQ ID NO: 2 - protein, 428 aa, Homo sapiens [sequence omitted]

SEQ ID NO: 3 - DNA, 1317 bp, Homo sapiens
  CDS (1)..(1317)
  ref <308>: DATABASE ACCESSION NUMBER: Genbank / AF038650
  ref <309>: DATABASE ENTRY DATE: 1999-03-25
  ref <313>: RELEVANT RESIDUES: (1)..(1317)
atggttcaatggaagagactctgccagctgcattacttgtgggctctgggctgctatatgctgctggccactgtggctctgaaactttctttcaggttgaagtgtgactctgaccacttgggtctggagtccagggaatctcaaagccagtactgtaggaatatcttgtataatttcctgaaacttccagcaaagaggtctatcaactgttcaggggtcacccgaggggaccaagaggcagtgcttcaggctattctgaataacctggaggtcaagaagaagcgagagcctttcacagacacccactacctctccctcaccagagactgtgagcacttcaaggctgaaaggaagttcatacagttcccactgagcaaagaagaggtggagttccctattgcatactctatggtgattcatgagaagattgaaaactttgaaaggctactgcgagctgtgtatgcccctcagaacatatactgtgtccatgtggatgagaagtccccagaaactttcaaagaggcggtcaaagcaattatttcttgcttcccaaatgtcttcatagccagtaagctggttcgggtggtttatgcctcctggtccagggtgcaagctgacctcaactgcatggaagacttgctccagagctcagtgccgtggaaatacttcctgaatacatgtgggacggactttcctataaagagcaatgcagagatggtccaggctctcaagatgttgaatgggaggaatagcatggagtcagaggtacctcctaagcacaaagaaacccgctggaaatatcactttgaggtagtgagagacacattacacctaaccaacaagaagaaggatcctcccccttataatttaactatgtttacagggaatgcgtacattgtggcttcccgagatttcgtccaacatgttttgaagaaccctaaatcccaacaactgattgaatgggtaaaagacacttatagcccagatgaacacctctgggccacccttcagcgtgcacggtggatgcctggctctgttcccaaccaccccaagtacgacatctcagacatgacttctattgccaggctggtcaagtggcagggtcatgagggagacatcgataagggtgctccttatgctccctgctctggaatccaccagcgggctatctgcgtttatggggctggggacttgaattggatgcttcaaaaccatcacctgttggccaacaagtttgacccaaaggtagatgataatgctcttcagtgcttagaagaatacctacgttataaggccatctatgggactgaactttga

SEQ ID NO: 4 - protein, 438 aa, Homo sapiens
MVQWKRLCQLHYLWALGCYMLLATVALKLSFRLKCDSDHLGLESRESQSQYCRNILYNFLKLPAKRSINCSGVTRGDQEAVLQAILNNLEVKKKREPFTDTHYLSLTRDCEHFKAERKFIQFPLSKEEVEFPIAYSMVIHEKIENFERLLRAVYAPQNIYCVHVDEKSPETFKEAVKAIISCFPNVFIASKLVRVVYASWSRVQADLNCMEDLLQSSVPWKYFLNTCGTDFPIKSNAEMVQALKMLNGRNSMESEVPPKHKETRWKYHFEVVRDTLHLTNKKKDPPPYNLTMFTGNAYIVASRDFVQHVLKNPKSQQLIEWVKDTYSPDEHLWATLQRARWMPGSVPNHPKYDISDMTSIARLVKWQGHEGDIDKGAPYAPCSGIHQRAICVYGAGDLNWMLQNHHLLANKFDPKVDDNALQCLEEYLRYKAIYGTEL

SEQ ID NO: 5 - DNA, 2128 bp, Homo sapiens
  CDS (354)..(1670)
  ref <308>: DATABASE ACCESSION NUMBER: GenBank / AF102542
  ref <309>: DATABASE ENTRY DATE: 1999-01-31
  ref <313>: RELEVANT RESIDUES: (1)..(2128)
cttgagaatgtttctgcatgatttctctgggcttgctgtcttacctgagagcaggaagtcaaaaggagagtgatggagccacctgctgccctctactttgcagatattaaagaggagcctgaaactgttccttggacatcttatgaatgtcagaaaataccttttggagggttagaagatcaggggacatggttgttcacatttgctgccacggaacaccgccagtcttcacttggaaacagaatcacgccttgtgaagagatcatccctaagcaggagagaagctactaaaggattgtgtcctcctccaccttccctgtgctcggtctccacctgtctcccattctgtgacgatggttcaatggaagagactctgccagctgcattacttgtgggctctgggctgctatatgctgctggccactgtggctctgaaactttctttcaggttgaagtgtgactctgaccacttgggtctggagtccagggaatctcaaagccagtactgtaggaatatcttgtataatttcctgaaacttccagcaaagaggtctatcaactgttcaggggtcacccgaggggaccaagaggcagtgcttcaggctattctgaataacctggaggtcaagaagaagcgagagcctttcacagacacccactacctctccctcaccagagactgtgagcacttcaaggctgaaaggaagttcatacagttcccactgagcaaagaagaggtggagttccctattgcatactctatggtgattcatgagaagattgaaaactttgaaaggctactgcgagctgtgtatgcccctcagaacatatactgtgtccatgtggatgagaagtccccagaaactttcaaagaggcggtcaaagcaattatttcttgcttcccaaatgtcttcatagccagtaagctggttcgggtggtttatgcctcctggtccagggtgcaagctgacctcaactgcatggaagacttgctccagagctcagtgccgtggaaatacttcctgaatacatgtgggacggactttcctataaagagcaatgcagagatggtccaggctctcaagatgttgaatgggaggaatagcatggagtcagaggtacctcctaagcacaaagaaacccgctggaaatatcactttgaggtagtgagagacacattacacctaaccaacaagaagaaggatcctcccccttataatttaactatgtttacagggaatgcgtacattgtggcttcccgagatttcgtccaacatgttttgaagaaccctaaatcccaacaactgattgaatgggtaaaagacacttatagcccagatgaacacctctgggccacccttcagcgtgcacggtggatgcctggctctgttcccaaccaccccaagtacgacatctcagacatgacttctattgccaggctggtcaagtggcagggtcatgagggagacatcgataagggtgctccttatgctccctgctctggaatccaccagcgggctatctgcgtttatggggctggggacttgaattggatgcttcaaaaccatcacctgttggccaacaagtttgacccaaaggtagatgataatgctcttcagtgcttagaagaatacctacgttataaggccatctatgggactgaactttgagacacactatgagagcgttgctacctgtggggcaagagcatgtacaaacatgctcagaacttgctgggacagtgtgggtgggagaccagggctttgcaattcgtggcatcctttaggataagagggctgctattagattgtgggtaagtagatcttttgccttgcaaattgctgcctgggtgaatgctgcttgttctctcacccctaaccctagtagttcctccactaactttctcactaagtgagaatgagaactgctgtgatagggagagtgaaggagggatatgtggtagagcacttgatttcagttgaatgcctgctggtagcttttccattctgtggagctgccgttcctaataattccaggtttggtaacgtggaggagaactttgatggaaagagaacttccttctgtactgttaacttaaaaataaatactcctgatcaaaaaaaaaaaa

SEQ ID NO: 6 - protein, 438 aa, Homo sapiens; identical to SEQ ID NO: 4

SEQ ID NO: 7 - DNA, 3435 bp, Homo sapiens
  CDS (862)..(2223)
  ref <308>: DATABASE ACCESSION NUMBER: GenBank / AF132035
  ref <309>: DATABASE ENTRY DATE: 2000-04-08
  ref <313>: RELEVANT RESIDUES: (1)..(3435)
tcaatgcactgttcaaggatgtttgagagattttctgtcttgcctgaatacaaaacactgtagggaacctcttgtgcttgactttgcacacacacgggatcttcagggtgggtacatttaaaactgacaagtattgccaaattgccccccaacgtagtttttacaccccacctactactctcagtgaaaaatcaggccctttttattttaagggctaaattgctaatccctgtgtgaaaatgggtgttaacaaaataaaaaataaggaaaatatgcaggcttacccaattttgttatctatacattttacagtctctaatacttagcatagctcctttgggtgtctgcccaccacccttctttctgaacgtggggttccctgggctttccttgtttgacttcagacctggatctaatagggatctggaggtagaatcaagacgaatgttccctttggccttaggtgagtcctttgcatctctgaggccctgttgatgacatccagttctctagacctctgcccaaaccagagcaatccctcctcaaatatgctaagcagaaatgcagacaatataagacacactatatttatattttaggagtttacattaccttcccttctgtttggaaaccaacatctctcaaaattaggtatagttaacacagaataccccaaaataccaccattcttcataattccatttccttatttctggattctttcaagggtaaaaaccttgtttcattcatctttggggcagcatttgcctagtatgtggtaggcgcccagtagattttggataaactgacctgattttctgttaatattccaactggattatttctttctcctcttacagaatgaagatattcaaatgttattttaaacataccctacagcagaaagttttcatcctgtttttaaccctatggctgctctctttgttaaagcttctaaatgtgagacgactctttccgcaaaaagacatttacttggttgagtactccctaagtacctcgccttttgtaagaaacagatacactcatgttaaggatgaagtcaggtatgaagttaactgttcgggtatctatgaacaggagcctttggaaattggaaagagtctggaaataagaagaagggacatcattgacttggaggatgatgatgttgtggcaatgaccagtgattgtgacatttatcagactctaagaggttatgctcaaaagcttgtctcaaaggaggagaaaagcttcccaatagcctattctttggttgtccacaaagatgcaattatggttgaaaggcttatccatgctatatacaaccagcacaatatttactgcatccattatgatcgtaaggcacctgataccttcaaagttgccatgaacaatttagctaagtgcttctccaatattttcattgcttccaaattagaggctgtggaatatgcccacatttccagactccaggctgatttaaattgcttgtcggaccttctgaagtcttcaatccagtggaaatatgttatcaacttgtgtgggcaagattttcccctgaagtcaaattttgaattggtgtcagagttgaaaaaactcaatggagcaaatatgttggagacggtgaaacccccaaacagtaaattggaaagattcacttaccatcatgaacttagacgggtgccttatgaatatgtgaagctaccaataaggacaaacatctccaaggaagcacccccccataacattcagatatttgttggcagtgcttattttgttttaagtcaagcatttgttaaatatattttcaacaactccatcgttcaagacttttttgcctggtctaaagacacatactctcctgatgagcacttttgggctaccttgattcgggttccaggaatacctggggagatttccagatcagcccaggatgtgtctgatctgcagagtaagactcgccttgtcaagtggaattactatgaaggctttttctatcccagttgtactggatctcaccttcgaagcgtgtgtatttatggagctgcagaattaaggtggcttatcaaagatggacattggtttgctaataaatttgattctaaggtggaccctatcttgattaaatgcttggcagaaaagcttgaagaacagcagagagactggatcactttgccctcagaaaagttatttatggatagaaatctcactaccacatcatgatagtaaaatcaggatggaaataagagggtgcctgataaatggagtcagtgtggaattgaataccatactatgcccaatactgtttaaactcagtcctcccatattttaaaaggtgtccaaaattccatacacaagggaaagtgatctagcctttgatgttattagcctgcagttggctaggtttttttaatatttgtttttgcttgtaatctcactgagccaaatcagagatcttaaacattcagtcagtcatcaaacattattgagcacctaactatatgacaggcacttttttagagactgcggcttatcctcatcatagcaacctcggtatctttaagttctccacataacagtcaggattctactgaagaagcttttgaagtttgtggtaatcgtctgatcatataaaccacccatttcagagtagtgtttaagtactgtgaccaacactccacttgtctcttaactcagctttcaagacattccttaaccatcagagcagaggaggaaagactcactacctcagaaaaatctcaaagaatagtccaatttcctgcttgccaaagcataatctgccttttggtgcattacttggtcaattcagggttgaggagactgttggggggcatttataatgtatgaaagttaaggaagggggtgaggatgtgggttgggacaggtagtacctaaagaggagcgaagggatttatacaacatttttatcatgttacaaaacagtatcatgaatggcctcctttttagttcaactgtttctttaaaatgcatttactgattaaaaataagaactgaccaatagctccaagtgtcacacaccagaacattaagcctaagtcctcaattcataagttatcattctagacaagtttctttacaatggaatactttctatggaatcattataattctgttgtgtggaactatcaagctaagagtcactaaactttcttgaaaaggttgtgtgaaatatgacagctttctaaattaatttgtatagtcatttaaaattttttcttctctggcaactgtccaactggaatccagatttaaggtgataaaagctctaagtttcttgcagtctttttctcagcttagttccagagagaaaaaagctaattttcctaaggacacagcaagaatattcattaaggatattttctaaaacccacacttgagaaaaccacccaatga

SEQ ID NO: 8 - protein, 453 aa, Homo sapiens
MKIFKCYFKHTLQQKVFILFLTLWLLSLLKLLNVRRLFPQKDIYLVEYSLSTSPFVRNRYTHVKDEVRYEVNCSGIYEQEPLEIGKSLEIRRRDIIDLEDDDVVAMTSDCDIYQTLRGYAQKLVSKEEKSFPIAYSLVVHKDAIMVERLIHAIYNQHNIYCIHYDRKAPDTFKVAMNNLAKCFSNIFIASKLEAVEYAHISRLQADLNCLSDLLKSSIQWKYVINLCGQDFPLKSNFELVSELKKLNGANMLETVKPPNSKLERFTYHHELRRVPYEYVKLPIRTNISKEAPPHNIQIFVGSAYFVLSQAFVKYIFNNSIVQDFFAWSKDTYSPDEHFWATLIRVPGIPGEISRSAQDVSDLQSKTRLVKWNYYEGFFYPSCTGSHLRSVCIYGAAELRWLIKDGHWFANKFDSKVDPILIKCLAEKLEEQQRDWITLPSEKLFMDRNLTTTS

SEQ ID NO: 9 - DNA, 26 bp, Artificial
  Sense primer for cloning hC2GnT-M
  primer_bind (1)..(26)
cggaattcgacgatggttcaatggaa

SEQ ID NO: 10 - DNA, 25 bp, Artificial
  Antisense primer for cloning hC2GnT-M
  primer_bind (1)..(25)
gctctagagccaagttcagtcccat